(19) United States
(12) Patent Application Publication
(10) Pub. No.: US 2015/0232876 A1
(43) Pub. Date: Aug. 20, 2015

(54) Use of a Receptor Kinase Having LysM Motifs in Order to Improve the Response of Plants to Lipochitooligosaccharides

(71) Applicants: INSTITUT NATIONAL DE LA RECHERCHE AGRONOMIQUE, Paris (FR); CENTRE NATIONAL DE LA RECHERCHE SCIENTIFIQUE, Paris (FR); UNIVERSITE PAUL SABATIER (TOULOUSE III), Toulouse Cedex 09 (FR)

(72) Inventors: Jean-Jacques Bono, Toulouse (FR); Judith Fliegmann, Toulouse (FR); Christophe Lachaud, Dundee Central Scotland (GB); Julie Cullimore, Castanet-Tolosan (FR); Marie Cumener, Auzeville-Tolosane (FR); Virginie Gasciolli, Toulouse (FR); Benoit Lefebvre, Toulouse (FR); Nikita Malkov, Toulouse (FR); Charles Rosenberg, Castanet Tolosan (FR)

(73) Assignees: INSTITUT NATIONAL DE LA RECHERCHE AGRONOMIQUE, Paris (FR); CENTRE NATIONAL DE LA RECHERCHE SCIENTIFIQUE, Paris (FR); UNIVERSITE PAUL SABATIER (TOULOUSE III), Toulouse Cedex 09 (FR)

(21) Appl. No.: 14/425,258

(22) PCT Filed: Aug. 30, 2013
(86) PCT No.: PCT/IB2013/058147

§ 371 Date: Mar. 2, 2015

(30) Foreign Application Priority Data
Aug. 30, 2012 (FR) 1258098

(51) Int. Cl. C12N 15/82; C12N 9/12

(52) U.S. Cl. CPC C12N 15/8262; C12N 9/12

(57) ABSTRACT

The invention relates to improving the response of a plant to lipochitooligosaccharidic Nod and Myc-LCO factors, by means of the expression, in the plant, of a polypeptide which has a sequence homology with the extracellular domain of the LysM-motif-containing receptor kinase LYR3 in Medicago truncatula and which is capable of binding to lipochitooligosaccharides with an increased affinity.

The invention relates to improved effects on plants of lipochitooligosaccharidic Nod and Myc-LCOs factors.

A very large number of terrestrial plants are able to establish, through the root system, symbiosis with soil microorganisms.

The best known of these endosymbiotic systems is the one which occurs between soil bacteria, collectively referred to as Rhizobia or Rhizobium, and roots of plants of the legume family, and leads to the formation of root nodules in which atmospheric nitrogen is fixed. Rhizobium-legume symbiosis is relatively specific: a particular species of Rhizobium does nodule only some legume species, whose number varies according to the species of Rhizobium concerned.

Another important type of root endosymbiosis is the symbiosis arbuscular endomycorrhizal, which occurs not only in legumes, but in most terrestrial plants, and involves fungi belonging to the order of Glomales.

The fungus provides to the plant inorganic compounds, mainly phosphate and nitrogen, and the plant provides carbohydrates produced by photosynthesis.

Rhizobium-legume symbiosis and endomycorrhizal symbiotic arbuscular have a number of commonalities. One of these is the nature of the symbiotic signals produced by the Rhizobia (Nod factors) or fungi (LCOs-Myc factors); indeed, in both cases, it is lipochitooligosaccharides (LCOs), composed of a skeleton of oligochitine having three to five residues of N-acetyl glucosamine linked by β (1-4), acylated on the nonreducing terminal glucosamine by a fatty acid (GOUGH & CULLIMORE, Molecular Plant-Microbe Interactions, 24, 867-78, 2012).

Among the Nod factors, the oligochitin backbone carries, in addition, various substituents at the reducing and nonreducing ends (acetylation, sulfation, methylation, fucosylation . . . ), and the length, degree of unsaturation, and the position of unsaturated bonds of the chain fatty acid may vary (DÉNARIÉ et al, Annu Rev Biochem, 65, 503-35, 1996. HAEZE & D'HOLSTERS, Glycobiology, 12, 79R-105R, 2002). This diversity of substituents results of the presence in different strains of Rhizobia, of different ranges of nod genes involved in these substitutions, and results in a wide variety of different factors Nod, which is involved in the specificity of the Rhizobium-legume interaction.

Myc-LCOs factors characterized to date have a much more limited variety of substituents: the fatty acid of the non-reducing end is mainly oleic acid (C18:1) or palmitic (C16:0), and the only other alternative being observed is a 0-sulfation of the reducing end (MAILLET et al, Nature, 469, 58-63, 2011).

Besides their roles in Rhizobium-legume symbioses, Nod factors and Myc-LCOs factors have a beneficial effect on the growth and development of plants. For example, it has been reported that Nod factors and Myc-LCOs factors improved development and root structure (OLAH et al, Plant J, 44, 195-207, 2005; MAILLET et al, Nature, 469. 58-63, 2011), and they favored germination, growth of various plants and photosynthetic yield (MAJ et al, J Chem Ecol, 35, 479-87, 2009; KIDAJ et al, Res Microbiol, 167, 144-50, 2012; PRITHIVIRAJ et al, Planta, 216, 437-45, 2003; SULEIMANOV et al, J Exp Bot, 53, 1929-1934, 2002; KHAN et al, J Plant Physiol, 165, 1342-1351, 2008).

N-acetyl glucosamine is also a core component of signaling molecules that are important in plant-pathogen interactions, and are particularly potent elicitors of defense responses in plants towards bacteria and pathogenic fungi. These are chitooligosaccharides (COs), which represent fragments of chitin released primarily by hydrolysis of the fungi cell wall, and which consist of a linear chain of residues of N-acetyl glucosamine linked by β (1-4) and having no substitution of a fatty acid, and bacterial peptidoglycan, the polysaccharide moiety of which is a copolymer of N-acetyl glucosamine and N-acetyl-muramic acid.

Proteins known to be involved in the perception of chitin fragments or bacterial peptidoglycans include Lys motifs protein (Lys-M), and especially proteins belonging to the LysM motif receptor kinase (LysM-RLKs for “Receptor LysM-Like Kinases”) family.

The LysM pattern (Pfam PF01476) is present in a great number of eukaryotic or prokaryotic proteins (BUIST et al. Molecular Microbiology, 68, 838-47, 2008). In prokaryotes, it is, for example, found in parietal enzymes involved in degradation of peptidoglycan; in eukaryotes, it is present in some chitinases, or receptors involved in the collection of molecules containing N-acetyl glucosamine.

The LysM motifs receptor kinases, whose existence has only been described in plants (ZHANG et al., Plant Physiology, 144, 623-36, 2007), include an extracellular domain containing 1 to 3 LysM motifs associated to an intracellular kinase domain via a transmembrane domain. In a number of these receptors, the intracellular kinase domain is inactive, that is to say, is not able to catalyze a phosphorylation reaction.

The analyzes from the sequences available in the databases showed that the LysM motifs receptor kinases belong to a multigene family which number of representatives depends on the species concerned. 17 members of this family were identified in Medicago truncatula (ARRIGHI et al., Plant Physiology, 142, 265-79, 2006) and in Lotus japonicus (LOHMANN et al., Mol Plant Microbe Interact, 23, 510-21. 2010), 12 in soybean, 5 in Arabidopsis thaliana, and 10 in rice (ZHANG, 2007, supra), but the precise function of most of them has not yet been elucidated.

It has been shown that some of these receptors are involved in the perception of COs chitin derivatives, and in the induction of the response of the plant to fungal infections; for instance in Arabidopsis thaliana LYK1/CERK1 (MIYA et al. Proc Natl Acad Sci USA, 104, 19613-18, 2007; WAN et al, Plant Cell 20, 471-81, 2008) and RLK4 (WAN et al, Plant Physiol, preprint online, Jun. 28, 2012.); and in rice, OsCERK1 (SHIMIZU et al, The Plant Journal, 64, 204-14, 2010). In rice and M. truncatula, a LysM motif protein has also been described as being involved in the perception of COs, however, this protein is devoid of kinase domain (U K A et al, Proc Natl Acad Sci USA, 103, January 11086-91, 2006; FLIEGMAN et al, Plant Physiology and Biochemistry 49, 709-720, 2011).

Other kinase motif LysM receptors have been described as involved in the perception of Nod factors, such as NFP and LYK3 in Medicago truncatula and NFRL NFR5 in Lotus japonicus (LIMPENS et al, Science, 302, 630.-3, 2003 MADSEN et al, Nature, 425, 637-40, 2003; RADUTOIU et al, Nature, 425, 585-92, 2003a; ARRIGHI et al, Plant Physiology, 142, 265-79, 2006; SMIT et al., Plant Physiol, 145, 183-91, 2007). It was also reported that Medicago truncatula NFP and a related protein from Parasponia andersonii (PaNFP) were also involved in the perception of Myc factors (MAILLET et al, Nature, 469, 58-63, 2011; OP DEN CAMP et al., Science, 331, 909-12, 2011).

Recently, it was reported that NFR1 and NFR5 from Lotus japonicus could bind directly to LCOs with high affinity (BROGHAMMER et al., Proc Natl Acad Sci USA, 109, 13859-64, 2012).

However, no direct interaction of NFP or LYK3 from Medicago truncatula with LCOs have been reported so far. Even though biochemical studies with radiolabeled Nod factors have shown the existence of three binding sites of Nod factors (NFBS referred to “Nod Factor Binding Site”) in the fractions obtained from roots of Medicago truncatula, or cultured cells of Medicago truncatula and Medicago varia (BONO et al, Plant J, 7, 253-60, 1995. GRESSENT et al, Proc Natl Acad Sci USA, 96, 4704-9, 1999. HOGG et al. Plant Physiol., 140, 365-73, 2006). These sites differ from each other in their affinity and selectivity towards the main Nod factor (NodSm-IV (Ac, S, C16: 2A2, 9)) produced by Sinorhizobium meliloti, the symbiont of Medicago, but they all recognize sulfated and non-sulfated Nod factors, and are independent of NFP.

One of these sites, referred to NFBS2, which has a high affinity for the NodSm factor is associated with the plasma membrane of cell of Medicago varia in culture (GRESSENT et al. Proc Natl Acad Sci USA, 96, 4704-9, 1999), and a site having similar properties has also been described in the membrane fraction of the cell cultures of Medicago truncatula (HOGG et al. Plant Physiol., 140, 365-73, 2006). However, the binding protein corresponding to NFBS2 had so far not been identified.

The inventors have now succeeded in identifying this protein as being the LYR3 protein, previously identified in silico by ARRIGHI et al. (2006), among LysM motifs receptor kinases, and also called MtLYK12 (ZHANG, 2007 previously cited), but to which no specific function could have been assigned. The inventors have further characterized its LCOs binding properties.

The present invention relates to the use of a polynucleotide selected from:
A polynucleotide encoding a LysM motifs receptor kinase whose extracellular domain flanked by the signal peptide has at least 45%, and by order of increasing preference, at least 50, 55, 60, 65, 70, 75, 80, 85, 90, 95, or 98% sequence identity, and at least 65%, and by order of increasing preference, at least 70, 75, 80, 85, 90, 95 or 98% sequence similarity with the region 1-274 of LYR3 of Medicago truncatula protein sequence of SEQ ID NO: 2;
A polynucleotide encoding a polypeptide comprising the extracellular domain of said LysM motifs receptor kinase;
   to improve the response of a plant to at least one LCO selected from Nod factors and factor Myc-LCOs.

Identity and sequence similarity values given here are calculated using the Needle program of the EMBOSS suite with default settings on a comparison window consists of the 1-274 amino acid SEQ ID NO: 1 and the region homologous to the target protein.

One means here by Nod factor or factor Myc-LCO, any LCO whose generic structure core comprises a chain of residues of N-acetyl glucosamine linked by β (1-4) and which is N-acylated at the non-reducing terminal glucosamine by a fatty acid. Said LCO can be of natural origin (provided by symbionts microorganisms or purified from them), or be produced by chemical synthesis and/or by genetic engineering. Numerous Nod and Myc-LCO factors are known by themselves, are for example the Nod factors described in review articles DENARIE et al, (1996) or D'HAEZE & HOLSTERS (2002, previously cited), as well as in PCT Applications WO 91/15496, WO 94/00466, or WO 2005/063 784, or the Myc factors described in the PCT application WO 2010/049817.

The skilled person can easily identify the extracellular domains of LysM motif receptor kinase used in the context of the present invention, based on their sequence homology with the sequence of protein LYR3 of Medicago truncatula, and further on the basis their affinity for LCOs and their binding selectivity to LCOs compared to Cos.

Examples of LysM motifs receptor kinases are listed in Table I below. Those whose extracellular domain has at least 45% identity and at least 65% sequence similarity with that of Medicago truncatula LYR3 protein are shown in bold with asterisks.

TABLE I

| Squence access | | Protein | Complete protein | | Extracellular domain | |
|---|---|---|---|---|---|---|
| number | Organism | name | Identity % | Similarity % | Identity % | Similarity % |
| Medtr5g019040 | Medicago | MtNFP | 30.5 | 49.7 | 29.5 | 48.6 |
| Medtr8g078300 | truncatula | MtLYR1 | 27.4 | 49.0 | 22.0 | 47.9 |
| ARRIGHI et al. (2006) | | MtLYR2 | 37.6 | 57.3 | 41.2 | 57.0 |
| Medtr5g019050 and | | MtLYR3* | 100 | | | |
| SEQ ID NO: 2 | | | | | | |
| Medtr5g085790 | | MtLYR4 | 39.1 | 57.4 | 40.0 | 56.5 |
| Medtr7g079350 | | MtLYR5 | 30.3 | 49.5 | 34.1 | 52.7 |
| Medtr7g079320 | | MtLYR6 | 30.6 | 48.1 | 35.6 | 53.1 |
| Medtr3g080170 | | MtLYR7 | 38.7 | 57.6 | 37.5 | 54.0 |
| Medtr5g086130 | | MtLYK3 | 26.4 | 44.7 | 17.9 | 33.1 |
| chr2.CM0545.250.r2.m | Lotus | LjNFR1a | 25.8 | 43.8 | 17.9 | 30.8 |
| chr2.CM0323.400.r2.d | japonicus | LjNFR5 | 31.2 | 49.1 | 28.0 | 45.1 |
| chr2.CM0323.420.r2.d | | LjLYS12* | 68.2 | 79.1 | 70.6 | 78.5 |
| SEQ ID NO: 4 | Glycine max | GmLYR3-1* | 66.8 | 80.9 | 71.7 | 84.1 |
| SEQ ID NO: 6 | | GmLYR3-11* | 66.7 | 80.4 | 71.8 | 84.1 |
| SEQ ID NO: 8 | Phaseolus | PvLYR3* | 64.7 | 78.9 | 66.7 | 80.1 |
| | vulgaris | | | | | |
| SEQ ID NO: 10 | Pisum | PsLYR3* | 80.2 | 89.2 | 84.3 | 91.5 |
| | sativum | | | | | |
| Ppa002539m et | Prunus | PpLYR3* | 53.3 | 68.3 | 54.2 | 68.6 |
| SEQ ID NO: 12 | persica | | | | | |
| Solyc02g089900 | Solanum | SILYR3* | 51.7 | 67.1 | 49.6 | 66.5 |
| | lycopersicum | | | | | |
| At3g21630 | Arabidopsis | AtCERK1 | 27.1 | 40.4 | 21.9 | 35.3 |
| At1g51940 | thaliana | AtLysM-RLK2 | 27.1 | 43.6 | 21.6 | 34.8 |
| At2g33580 | | AtLysM-RLK3 | 38.0 | 57.3 | 36.7 | 51.7 |
| At2g23770 | | AtLysM-RLK4 | 41.2 | 62.5 | 40.8 | 62.8 |
| At3g01840 | | AtLysM-RLK5 | 24.1 | 43.1 | 22.8 | 43.2 |

Reference Sources:
Medicago truncatula-Genome Sequencing Project Release version: Mt3.5.1 (http://medicagohapmap.org/) Lotus japonicus genome assembly build 2.5 (http://www.kazusa.or.jp/lotus/), LOHMANN et al (2010, supra), Arabidopsis thaliana-The Arabidopsis Information Resource (TAIR) (http://www.arabidopsis.org/);
Solanum lycopersicum-3.0.1 genome (http://solgenomics.net/organism/Solanum_lycopersicum/genome) Prunus persica (peach)-Phytozome v9.1 (http://www.phytozome.org).

Typically, the extracellular domain of a LysM motifs receptor kinase used according to the invention is capable of binding to at least one LCO selected from Nod factors and Myc-LCO factors, with a dissociation constant (Kd), less than or equal to 100 nM, preferably less than or equal to 50 nM, and most preferred manner less than or equal to 20 nM.

Said extracellular domain is capable of binding to at least one LCO selected from Nod factors and Myc-LCO factors with a binding affinity at least 50 times, preferably at least 100 times, preferably at least 200 times, and so most preferably at least 500 times greater than its binding affinity for the corresponding CO is (that is to say with a CO having the same oligochitin backbone than the said Nod factor or the said Myc-LCO factor).

In the case of a polypeptide comprising the extracellular domain of said LysM motifs receptor kinase, it may be constituted by the isolated extracellular domain, or comprise one or more other domains, such as a transmembrane domain (or a membrane anchor sequence) and optionally an intracellular kinase domain (which may either have a kinase activity, or not). These may be optionally a chimeric polypeptide, wherein the domain(s) associated to the extracellular domain derived from one or more other(s) protein(s) that the LysM motifs receptor kinase from which said extracellular domain results. For example, the transmembrane domain and optionally the intracellular kinase domain, can come from one or possibly two LysM motifs receptor kinase(s) which are different from that which the said extracellular domain results. This or these other(s) LysM motifs receptor kinase(s) may be derived from a plant of the same species from which the extracellular domain results, or from one or two plants of different species. The extracellular domain may also be combined with domains derived from proteins other than the LysM motifs receptor kinase(s), such as those described by LORENZO et al. FEBS Letters 585, 1521-1528, 2011, involved in defense responses, resistance to pathogens and plant productivity.

The present invention relates to a method for improving response of a plant to at least one LCO selected from Nod factors and Myc-LCO factors, characterized in that it comprises transforming said plant with a polynucleotide encoding a LysM motif receptor kinase, or a polypeptide comprising the extracellular domain of said receptor, as defined above, and the expression of said receptor or said polypeptide in said plant.

Improving the response of a plant to Nod factors and/or Myc-LCO factors may result in an improvement of the capacity of nodulation and/or endomycorhization of said plant as well as in a better response to treatment with Nod factors and/or Myc-LCO factors, allowing to increase one or more of the beneficial effects of these factors, such as improving germination, root development, growth, photosynthetic yield or resistance to pathogens.

The process according to the invention can be implemented by the usual methods, known in themselves, from genetic engineering and plant transgenesis. Conventionally, it comprises the following steps:
Transforming a plant cell with a vector containing an expression cassette comprising a polynucleotide encoding a LysM motifs receptor kinase, or a polypeptide comprising the extracellular domain of said receptor, as defined above, under control transcription of a suitable promoter;
Culturing said transformed cell in order to regenerate a plant having in its genome a transgene containing said expression cassette.

Many techniques for transforming plant germinal or somatic cells, (isolated as a tissue culture or organ or whole plant), and regeneration of plants are known to those skilled in the art. The choice of the most appropriate method will generally depend on the plant concerned.

The present invention also relates to recombinant DNA constructs for the expression of a LysM motifs receptor kinase or a polypeptide comprising the extracellular domain of said receptor, as defined above.

This includes expression cassettes comprising a polynucleotide encoding said polypeptide or said receptor kinase, placed under the transcriptional control of a suitable promoter.

Said promoter may be the endogenous promoter of said LysM motifs receptor kinase, in which case, the expression cassette may be advantageously constituted by the sequence coding for said receptor kinase, flanked by 0.5 to 2 kb, preferably from 1 to 1.5 kb of genomic sequence upstream.

Alternatively, it may be a heterologous promoter.

A wide range of suitable promoters for the expression of genes of interest into plant cells or plants is available in the art.

These promoters may be obtained for example from plants, plant viruses, or bacteria such as Agrobacterium. They include constitutive promoters, i.e. promoters that are active in most tissues and cells and in most environmental conditions, as well as tissue-specific or cell-specific promoters, which are active solely or mainly in certain tissues or some cell types, and inducible promoters that are activated by physical or chemical stimuli.

Examples of constitutive promoters that are commonly used in plant cells are virus 35S promoter of the cauliflower mosaic (CaMV) described by KAY et al. (Science, 236, 4805, 1987), or its derivatives, virus promoter vein mosaic cassava (CsVMV) described in International Application WO 97/48819, the ubiquitin promoter from maize (CHRISTENSEN & QUAIL, Transgenic Res, 5, 213-8, 1996), trefoil (Ljubql, MAEKAWA et al. Mol Plant Microbe Interact. 21, 375-82, 2008) and Arabidopsis (UBQ10, Norris et al. Plant Mol. Biol. 21, 895-906, 1993) or the “actin-actin-Intron” promoter of rice (McElroy et al, Mol Gen Genet 231, 150-160, 1991; GenBank accession number S 44221).

Advantageously, promoter conferring a specific or favorite expression in the tissues of the root will be used; as non-limiting examples, the promoter of the maize allothionéine (DE FRAMOND et al, FEBS 290, 103.-106, 1991 Application EP 452269) can be mentioned as well as the chitinase promoter described by SAMAC et al (Plant Physiol 93, 907-914, 1990), the promoter of the glutamine synthetase soybean root described by HIREL et al. (Plant Mol. Biol. 20, 207-218, 1992), the promoter of rice RCC3 (PCT Application WO 2009/016104), the promoter of rice antiquitine (PCT Application WO 2007/076115), the promoter of the LRR receptor kinase described in PCT application WO 02/46439, the ZRP2 promoter of maize described in U.S. Pat. No. 5,633,363, the promoter LeExtl tomato (Bucher et al. Plant Physiol. 128, 911-923, 2002), the Arabidopsis promoter pCO2 (HEIDSTRA et al, Genes Dev. 18, 1964-1969, 2004).

The present invention also encompasses recombinant vectors, resulting from the insertion of an expression cassette in accordance with the invention in a host vector.

The expression cassettes and recombinant vectors according to the invention may, of course, also comprise other sequences, usually used in this type of construction. The choice of such sequences will be carried out in conventional manner, by one skilled in the art according to particular criteria such as the selected host cells, transformation protocols contemplated, etc.

Non-limiting examples, transcription terminators, leader sequences and polyadenylation sites may be mentioned. These sequences do not modify the specific properties of the promoter or gene which they are associated, but can improve overall qualitatively or quantitatively, transcription, and optionally, translation. Examples of such sequences frequently used in plants, may be mentioned among the most common, the terminator of the 35S RNA of CaMV, the terminator of the nopaline synthase gene, etc. In order to increase the level of expression, enhancer sequences may also be used (sequences “enhancer” of transcription and translation).

Among other sequences commonly employed in the construction of recombinant expression cassettes and vectors, sequences allowing the monitoring of transformation, and the identification and/or selection of transformed cells or organisms can be used. These include reporter genes, giving these cells or organisms an easily recognizable phenotype, or selection marker genes: only cells or organisms expressing a marker gene selection are viable under given conditions (selective conditions). Reporter genes are frequently used, for example that of the beta-glucuronidase (GUS), the luciferase, or that of the “green or red fluorescent protein” (GFP/RFP) and derivatives thereof. The selection marker genes are generally genes for resistance to an antibiotic, or also, in the case of plants or plant cells to an herbicide. There is a wide variety of selection marker genes including the skilled artisan can make their choices based on criteria that will itself be determined.

The choice of the appropriate vector will depend more particularly the intended host, and of the envisaged method for the transformation of the intended host. Many methods for the genetic transformation of plant cells or plants are available in the art for many plants, monocotyledonous or dicotyledonous. As non limiting examples, mention may be made of virus-mediated transformation, transformation by microinjection, by electroporation, microprojectile transformation, Agrobacterium transformation, etc.

The present invention also encompasses any host cell transformed with a polynucleotide coding for a LysM motif receptor kinase or a polypeptide comprising the extracellular domain of said receptor, as defined above, which includes in particular host cells transformed with an expression cassette or a recombinant vector according to the invention.

A cell or organism transformed with a polynucleotide means any cell or organism whose genetic content has been amended by transferring said polynucleotide in said cell or organism, whatever the transfer method used, and whether the information gene provided by said polynucleotide is integrated into chromosomal DNA or remains extra chromosomal.

Said host cell may be a prokaryotic or eukaryotic cell. In the case of a prokaryotic cell, it may especially be a cell such that Agrobacterium tumefaciens or Agrobacterium rhizogenes. In the case of a eukaryotic cell, it may include a plant cell, derived from a dicotyledonous or monocotyledonous plant. The construct can be expressed transiently, it may also be incorporated into a stable extrachromosomal replicon or integrated into the chromosome.

The present invention also relates to a transgenic plant comprising in its genome at least one copy of a transgene comprising a polynucleotide encoding a LysM motifs receptor kinase or a polypeptide comprising the extracellular domain of said receptor, as defined above.

Is defined herein as a transgenic plant transformed, a plant in which the genetic information provided by an exogenous transforming polynucleotide is stably integrated into the chromosomal DNA, as a transgene, and can be transmitted to progeny of said plant. So this definition also covers the descendants of plants resulting from the initial transgenesis, since they contain in their genome at least one copy of the transgene.

The plant material (protoplasts, callus, cuttings, seeds, etc.) obtained from the transformed cells or transgenic plants according to the invention is also part of the present invention. The invention also includes products obtained from transgenic plants according to the invention, including fodder, wood, leaves, stems, roots, flowers and fruits.

The present invention will be better understood with the help of the description which follows, which refers to non-limiting examples illustrating the characterization of LYR3 protein, and demonstrate its role in the perception of LCOs.

### EXAMPLE 1

### Characterization of a Binding Site of High Affinity for Lipochitooligosaccharides in Medicago truncatula

In Medicago varia and Phaseolus vulgaris, binding sites having high affinity for factors Nod produced by respective symbionts of these two plants have been previously described from the membrane fraction of the cultured cells, and respectively called MvNFBS2 and PvNFBS2 (GRESSENT et al, Proc Natl Acad Sci USA, 96, 4704-9, 1999. GRESSENT et al, Mol Plant Microbe Interact, 15, 834-9, 2002). MvNFBS2 and PvNFBS2 are enriched in the plasma membrane. However, the proteins corresponding to these NFBS2 sites had not been identified. More recently, a binding site with comparable properties, namely a high affinity for the S. meliloti Nod factor, NodSm-IV (Ac, S, C16:2Δ2, 9), has been identified in the cell membrane fraction in culture of Medicago truncatula (HOGG et al., Plant Physiol., 140, 365-73, 2006).

To further characterize this site, binding experiments to balance of different chitooligosaccharides (CO) or lipochitoologosaccharides (LCO) in the presence of radioligand LCO-IV (³⁵S, C16: 2Δ2, 9), representative of a S. meliloti Nod factor, were performed. The tested compounds show variations at the oligochitin backbone (number of sugar residues) and its substitutions (presence or absence of sulfate group) level as well as at the level of the fatty acid chain (chain length and number and type unsaturations) acylating the N-terminal non-reducing sugar of LCOs. The nomenclature used herein to refer to these compounds is the following: for the natural Nod factors, the indication Nod is followed by the abbreviated name of the bacterial species producing the concerned Nod factor; for synthetic molecules, CO denotes a chito-oligosaccharide, LCO a lipochitooligosaccharide. The number of sugar units is indicated by Roman numbers. The nature of the fatty acid chain of LCO, and the possible substitutions on the oligochitin backbone (Ac for O-acetyl, S for sulfate group) are mentioned in parentheses.

These experiments were performed with membrane fractions obtained from cultured cells of mutant dmi3 of Medicago truncatula (who is able to establish either rhizobial symbiosis or symbiotic arbuscular endomycorrhizal) because it has been observed that they were richer in binding sites MtNFBS2 than wild plants (HOGG et al., Plant Physiol., 140, 365-73, 2006).

The cultured cells were obtained from the roots of plants calli produced and harvested as described by GRESSENT et al. (Proc Natl Acad Sci USA, 96, 4704-9, 1999).

The cells were homogenized 6 times 5 seconds at 4° C. in a propeller mill in extraction buffer (25 mM Tris-HCl pH 8.5, 0.47 M sucrose, 10 mM EDTA, 10 mM dithiothreitol) with added cocktail of protease inhibitors (0.1 mM (2-Amino-ethyl)-4-benzenesulfonyl fluoride chlorohydrate (AEBSF), antipain, leupeptin, aprotinin, pepstatin, chymostatin) and the homogenate was treated as previously described by GRESSENT et al. (1999 previously cited). The membrane fraction sedimented at 45,000 xg, was resuspended in binding buffer (buffer 25 mM Na-cacodylate pH 6.0, 0.25 M sucrose, 1 mM MgCl₂, 1 mM CaCl₂, inhibitor cocktail proteases) adjusted to 50% glycerol and stored at −80° C.

For binding experiments at balance, from 10 to 50 micrograms of membrane protein fraction were incubated with 0.4, 0.7 or 2 nM of Nod factor labeled with ³⁵S, (LCO-IV (³⁵S, C16:2Δ2, 9)) in binding buffer (final volume 200 μl). The component of non-specific binding was determined in the presence of 2 μM LCO-IV (S, C16:2Δ2, 9).

The competition experiments were carried out in the presence of varying concentrations of competitors to determine their affinity (K_{d}, Kᵢ) for the binding site. All experiments were performed in triplicate.

The reaction mixtures were incubated for 1 hour at 0° C. in microtiter plates with 96 wells (Nunc), filtered to separate the free radioligand from the bound one, and the filters washed and their radioactivity measured as described previously by GRESSENT et al (1999). Data were analyzed using the software RADLIG, version 4 (Biosoft, Cambridge, UK).

The results of these experiments are presented in Table II below.

TABLE II

| Compound | Affinity (nM) |
|---|---|
| LCO-IV(S, C16:2Δ2, 9) | 15 |
| LCO-IV(C16:2Δ2, 9) | 15 |
| Myc-LCO: LCO-IV(S, C16:0) | 6 |
| Myc-LCO: LCO-IV(S, C18:1Δ9) | 10 |
| Myc-LCO: LCO-IV(C18:1Δ9) | 11 |
| LCO-II(C16:1Δ9) | >5000 |
| CO-IV | >5000 |

The Nod-factor LCO IV (S, C16: 2A2, 9) has a high affinity (K_{d}=15 nM) for the binding site and its non sulfated counterpart, LCO-IV (C16: 2A2, 9) (Ki=15 nM). The LCOs corresponding to Myc-LCOs factors: LCO-IV (S, C18: 1Δ9) and non-sulfated counterpart, and LCO-IV (S, C16:0) are also recognized with very high affinity (Ki from 6 to 11 nM). In contrast, LCO-II (C16: 1Δ9) and chitotetraose (CO-IV) have a very low affinity (Ki of 5 μM and Ki>2 μM, respectively).

The binding site present in Medicago truncatula has therefore affinity and selectivity characteristics similar to those previously observed for the website link MvNFBS2. It will hereinafter be named MtNFBS2.

### EXAMPLE 2

### Identification of a Polypeptide of 100 Kda Corresponding to MtNFBS2

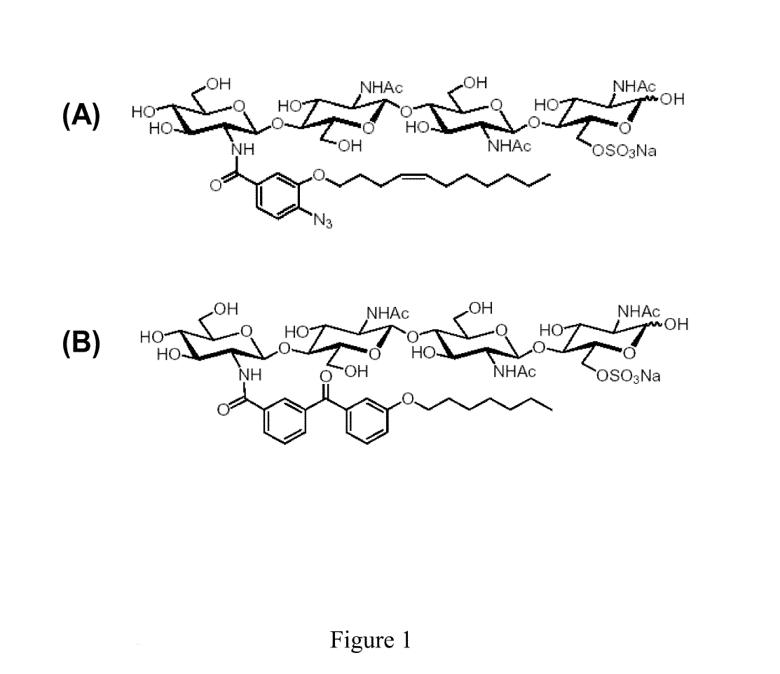

To assess the molecular mass of the affine protein of Nod factors responsible for binding properties of MtNFBS2, a photolabeling with Nod factors analogs containing a photoactivatable group, which after irradiation in a given wavelength reactive species which can form a covalent bond with the surrounding molecules, has been taken. The chemical structure of selected analogs is shown in FIG. 1. They contain a photoactivatable azido group (FIG. 1A) or benzophenone group (FIG. 1B), respectively activated at 254 and 380 nm, and it was verified that their affinity for MtNFBS2 was comparable to that of LCO-IV (S, C16:2Δ2, 9). Non-sulfated homologues of these molecules have been synthesized and labeled with ³⁵S to enable detection of the stable complex ligand/binding protein.

For photolabeling, 5 nM of radioactive and photoactivatable ligand, alone or in the presence of an excess (2 μM) different competitors were incubated in binding buffer, in the same conditions as described in Example 1 above for the binding experiments at equilibrium, with 300 micrograms of membrane protein fraction obtained from cultured cells of wild-type Medicago truncatula (A 17), or the dmi3 mutant of Medicago truncatula. After 1 hour of incubation, the samples were irradiated for 5 min at 254 nm or 10 min at 365 nm with a fluorescent tube at a distance of 4 cm. The free ligand was removed by centrifugation, and the membrane pellet was washed two times in binding buffer.

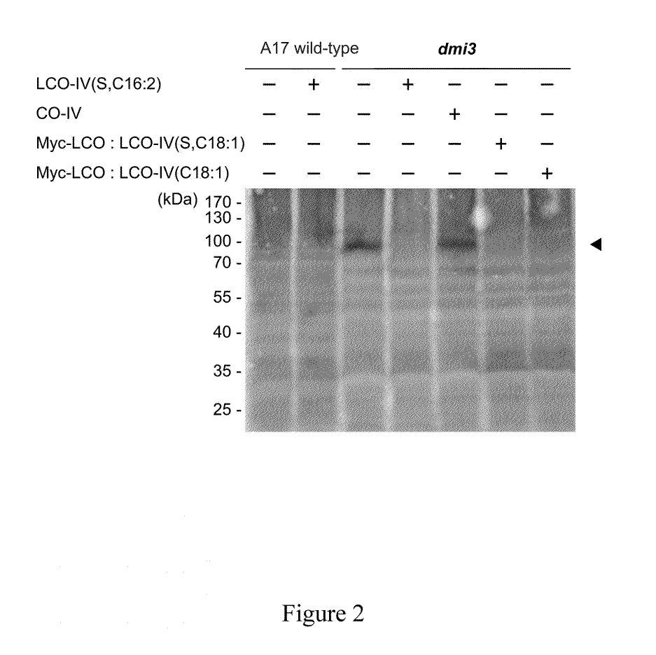

The membrane pellet proteins were separated by polyacrylamide gel electrophoresis in 10% SDS, and transferred onto nitrocellulose membrane, and detection was carried out by autoradiography. The autoradiograph with the ligand containing the azide group is shown in FIG. 2.

A protein of apparent molecular weight close to 100 kDa is detectable in the membrane fraction obtained from the cells of the dmi3 mutant. The binding of the radiolabeled ligand to this polypeptide is completely abolished when the incubation is conducted in the presence of an excess of LCO-IV (S, C16: 2Δ2, 9). The bonding is also inhibited in the presence of an excess of Myc-LCOs factors sulfated or non-sulfated (LCO-IV (S, C18: 1Δ9 and LCO-IV (C18: 1Δ9), but not in the presence of an excess of the corresponding chito-oligosaccharide (CO-IV). Lack of specific staining in the membrane fraction obtained from the cells of the wild plant suggests that the 100 kDa polypeptide is present in an amount less than the detection threshold.

The same results (not shown) were obtained with the radioactive ligand containing the benzophenone group.

The fact that the 100 kDa polypeptide is detected in the membrane fraction of cells of dmi3 in culture but not in that of wild type plants, that it selectively binds to LCOs compared with COs, and that it recognizes similarly LCOs whether sulfated or not, suggests that it corresponds to the protein involved in the MtNFBS2 site.

### EXAMPLE 3

### Identification and Characterization of a LYSM Receptor Kinase Corresponding to MtNFBS2

In order to identify candidate proteins may correspond to the 100 kDa polypeptide, proteomic and transcriptomic approaches were performed.

Several candidate proteins were identified on the basis of their apparent molecular weight and a greater level of expression in dmi3 cells in culture than those from wild type plants.

Among these proteins were the LysM motifs receptor kinases: LYR3, LYK9 and LYR6. As it is known that proteins of this family are involved in the collection of molecules containing a skeleton chitinique, these receptors were selected for further experiments.

Genes LYR3, LYK9 and LYR6 were inserted into a binary vector, fused in C-terminal with a fluorescent protein (Yellow Fluorescent Protein: YFP) and introduced into Nicotiana benthamiana leaves using agroinfiltration by Agrobacterium tumefaciens. Meanwhile, NFP and LYK3 genes which encode putative receptors LYR4 and Nod factors were inserted into the same vectors and expressed in the same conditions.

For the construction of binary vectors, the GATEWAY technology (Invitrogen) was used. For each of the selected genes, the complete coding sequence was amplified by PCR using primers specific for this sequence and containing attB1 and attB2 recombination sites, then introduced by BP recombination into the input vector pDONR 207 (INVITROGEN). The thus obtained recombinant pENTR clone was then recombined with the vector pBin19-35S-GW-YFP (FROIDURE et al, Proceedings of the National Academy of Sciences of the United States of America, 107, 15 281-86, 2010. CANONNE and al, Plant Cell, 23, 3498-511, 2011) for generating the binary vector used to transform plants. This vector was introduced by electroporation into the strain LBA4404 of Agrobacterium tumefaciens carrying the plasmid ternary pBBRvirGN54D (VAN DER FITS et al, Plant Molecular Biology, 43, 495-502, 2000).

The transformed bacteria were grown overnight at 28° C. in YEB medium (5 g/l Bacto peptone, 5 g/l beef extract, 5 g/l sucrose, 1 g/l yeast extract, 2 mM MgSO₄) supplemented with 10 μg/ml rifampicin, 40 mg/ml gentamycin, and 50 micrograms/ml kanamycin. They were then harvested, washed 3 times with 10 mM of 2-morpholino ethanesulfonic acid solution (MES)/10 mM MgCl₂ (pH 5.6), and incubated in the same solution supplemented with 1 mg/ml of acetosyringone to a final optical density (600 nm) of 0.25, for at least one hour before the infiltration in the sheets 2, 3, and 4 of 4 weeks old N. benthamiana plants.

The leaves were observed 72 h after agroinfiltration. For LYR3/YFP, NFP/YFP and LYK3/YFP recombinant proteins, a yellow fluorescence was observed at the periphery of the epidermal cells, suggesting a localization to the plasma membrane. For LYK9/YFP, LYR4/YFP and LYR6/YFP necrotic leaf lesions are observed, suggesting a hypersensitive response reaction-type, probably mediated by the kinase domain of these proteins, as already observed in the case other LysM domain receptor kinase (MADSEN et al., The Plant Journal, 65, 404-17, 2011).

As a consequence, chimeric constructs, in which the transmembrane domain and the kinase domain are replaced by the transmembrane domain and the inactive kinase domain of NFP, were performed for LYR3, LYR4, LYK9 and LYR6 proteins. For these constructs, the cloning method “Golden Gate” was used (ENGLER et al, PLOS One, 3 (11). E3647, 2008) and the chimeric gene construct was inserted into a binary vector pCAMBIA2200 (http://www.cambia.org; GenBank: AF234313.1), as amended, under the control of the 35S promoter. The introduction of the binary vector into the strain LBA4404 of Agrobacterium tumefaciens, and the agroinfiltration of N. benthamiana leaves were performed as described above, with the only difference that 25 g/ml of kanamycin (instead of 50) were used in the culture medium of the transformed bacteria.

The chimeric proteins expressed by these constructs contain, in their C-terminal portion, the transmembrane domain and the intracellular kinase domain of NFP, and in their N-terminal portion, the extracellular domain of LYR3, LYR4, LYK9 or LYR6. Their expression in N. benthamiana leaves did not cause hypersensitive response.

3 days after agro-infiltration, leaves of N. benthamiana expressing the different constructs were harvested and ground in liquid nitrogen. Membrane fractions were prepared from the ground material, using the same protocol as that described above for the preparation of microsomal fractions, except that 0.6% (w/v) of polyvinyl-polypyrrolidone were added to extraction buffer. After centrifugation at 3000×g, the supernatant was collected and centrifuged at 45000×g. The resulting (membrane fraction) pellet was resuspended in binding buffer, and stored at −80° C. in the presence of 10% or 50% glycerol, until use.

Membrane proteins were separated by 10% polyacrylamide gel electrophoresis in SDS, and transferred onto nitrocellulose membrane, and detection of recombinant proteins made using anti-GFP antibody. Meanwhile, the properties of specific binding to LCOs were assessed by equilibrium binding, as described in Example 1, using 0.8 to 2.4 nM of LCO-IV (³⁵S, C16:2Δ2, 9) +/−2 μM of unlabeled LCO-IV (S, C16: 2Δ2, 9), and 25 micrograms to 80 micrograms of membrane protein, depending of the expression level of the protein labeled with YFP.

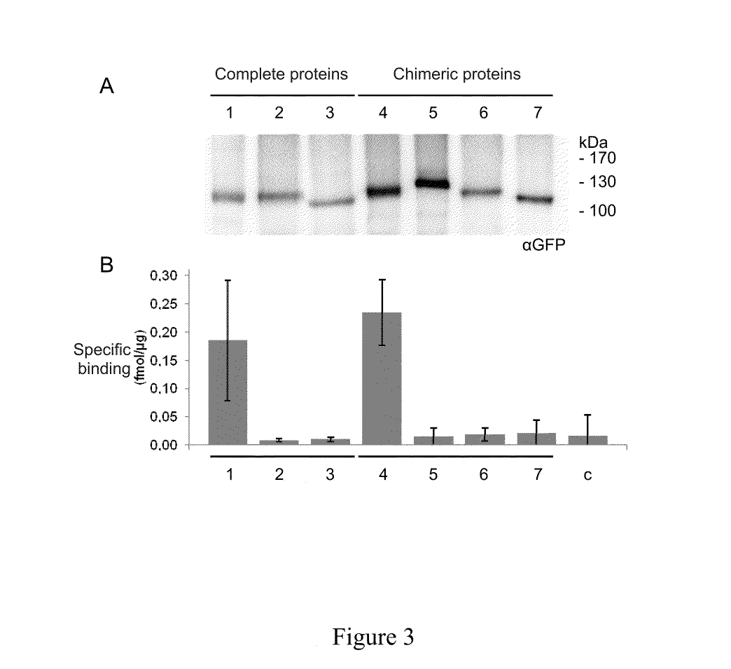

The results are illustrated in FIG. 3. 1. LYR3, 2. NFP, 3. LYK3, 4. LYR3-NFP, 5. LYR4-NFP, 6. LYR6-NFP, 7. LYK9-NFP, C: extract of unprocessed leaves.

These results confirm that all of the recombinant proteins are expressed (FIG. 3A), but that only those containing the extracellular domain of LYR3 bind the LCO ligand (FIG. 3B).

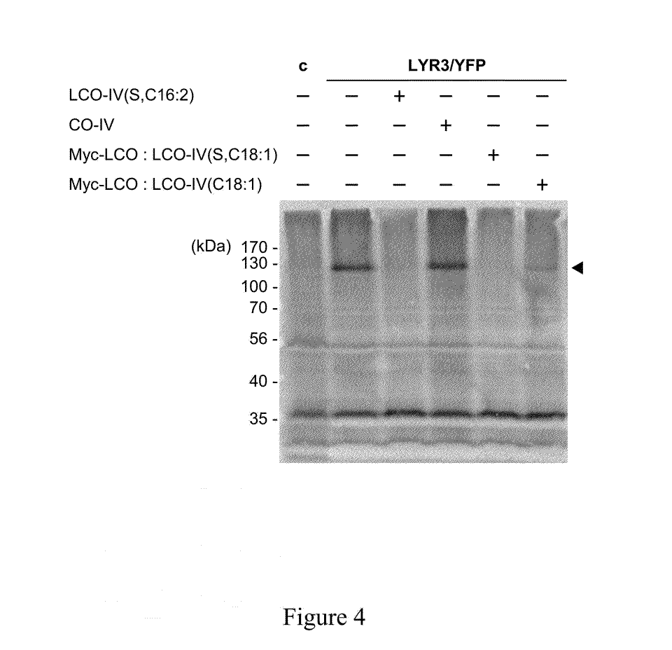

The direct binding of the LYR3 to LCOs was studied by photoaffinity labeling. 300 micrograms of protein from the membrane fraction of N. benthamiana leaves transformed with either the vector expressing the protein LYR3-YFP, or non-transformed (C) were incubated in the presence of radioactive ligand containing the photoactivatable and azido group in the presence or in the absence of competitor, using the protocol described in Example 2 above. The results are illustrated in FIG. 4.

These results demonstrate the selective labeling of a protein with an apparent molecular mass of 130 kDa corresponding to the expected molecular weight of the protein LYR3-YFP. The competition experiments performed in the presence of the LCO-IV (S, C16: 2Δ2, 9) Nod factor, sulfated or not sulfated, Myc-LCO (LCO-IV (S, C18:1Δ9) or LCO-IV (C18:1Δ9), and chitotetraose (CO-IV) show that, as for the MtNFBS2, binding is specific of LCOs compared with COs.

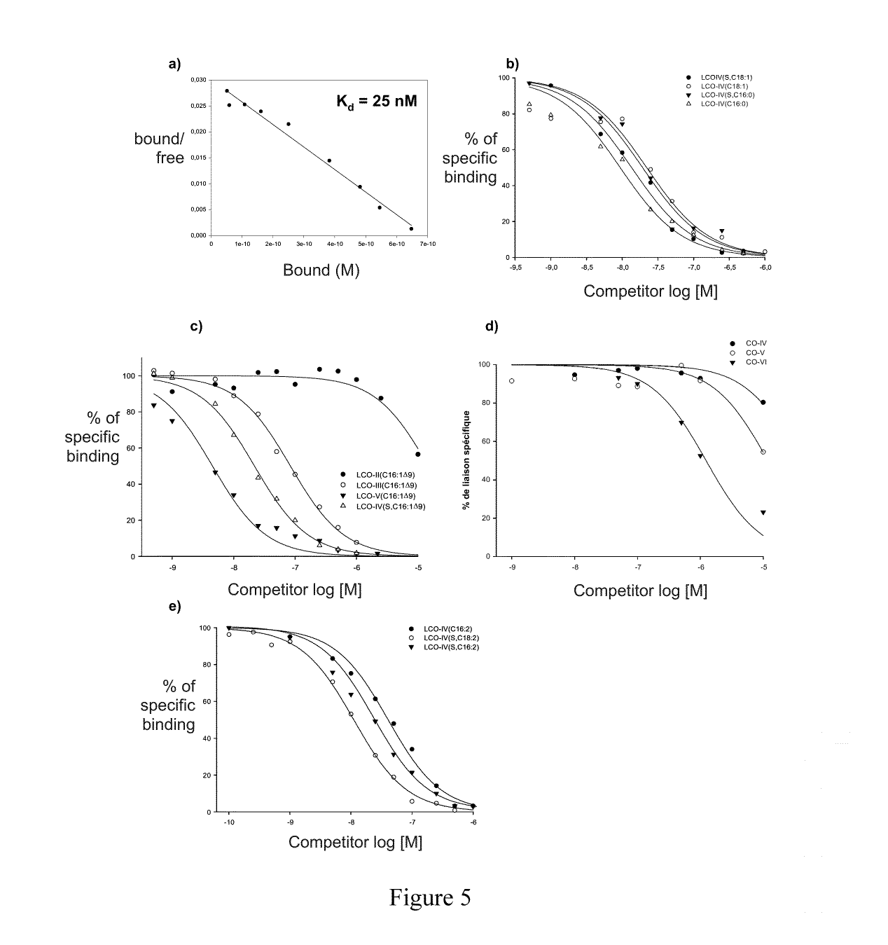

To further characterize the binding properties of the protein LYR3, saturation binding experiments using LCO-IV (³⁵S, C16:2Δ2, 9) and competitive binding experiments in the presence of different COs or LCOs were performed. The results are illustrated in FIG. 5 and in Table III below, which for comparison also shows the results observed in the same experiments with the MtNFBS2 site.

TABLE III

| | Affinity (nM) | Affinity (nM) for |
|---|---|---|
| Compounds | for LYR3 | MtNFBS2 |
| LCO-IV(S, C16:2Δ2, 9) | 25 +/− 3 | 15 +/− 2.5 |
| corresponding to the | | |
| radiolabeled homolog | | |
| LCO-IV(S, C18:2Δ2, 9) | 7 +/− 1.5 | 2 |
| Myc-LCO: LCO-IV(S, C16:0) | 26 +/− 5 | 6 |
| Myc-LCO: LCO-IV(S, C18:1Δ9) | 13.5 +/− 3 | 10 |
| LCO-IV(S, C16:1Δ9) | 21 +/− 2 | |
| Myc-LCO: LCO-IV(C16:0) | 15 +/− 2 | |
| Myc-LCO: LCO-IV(C18:1A9)) | 22 +/− 6 | 11 |
| LCO-IV(C16:2Δ2, 9) | 30 +/− 6 | 15 +/− 3 |
| LCO-II(C16:1Δ9) | >5000 | >5000 |
| LCO-III(C16:1Δ9) | 79 | |
| LCO-IV(S, C16:1Δ9) | 21 | |
| LCO-V(C16:1Δ9) | 4.3 | |
| CO-IV | >5000 | >5000 |
| CO-V | 12 000 | |
| CO-VI | 1200 | |

FIG. 5 shows the results of various experiments carried out with the LYR3-YFP protein expressed in the leaves of N. benthamiana: 5a): Scatchard plot of saturation binding experiment with LCO-IV (³⁵S, C16:2Δ2, 9), 5 b)-e) binding experiments with 0.9 nM LCO-IV (³⁵Δ2, 9) as the labeled ligand and varying concentrations of LCOs and COs competitors b) Myc-LCOs; c) LCOs with a variable number of GlcNAc residues d) COs with a variable number of GlcNAc residues; e) LCOs differing in the length of the fatty acid chain, and sulfated or not at the non-reducing terminal sugar.

Scatchard plot analysis showed the presence of a single class of binding sites with a Kd=25 nM for the Nod factor LCO-IV (S, C16: 2Δ2, 9), similar to that of MtNFBS2 (Kd=15 nM). All tested Myc-LCOs have similar affinity (FIG. 5b, Table II). The length of the oligosaccharide chain has an influence on the binding properties of the protein LYR3: the LCO-V has an affinity slightly higher than the LCO-IV, and the binding of the LCO-II is very low (FIG. 5c, Table III). All tested COs also have a very low affinity (FIG. 5d, Table III). Increasing the length of the fatty acid chain from C16:2 to C18:2 induce an increase of about 3 times in the affinity (FIG. 5e). However, LYR3 does not show significant selectivity towards the structure of the fatty acid chain, because LCOs with C16 chains containing 0, 1 or 2 unsaturated bonds have similar affinity (Table III) and the affinity differences between the Myc-LCOs (C16:0 or C18:1Δ9) and Nod factors (C16:2, Δ2, 9), which carry different fatty acid chains, are low (less than 2 times). Similarly, sulfation on the non-reducing terminal sugar does not significantly affect the binding properties (FIG. 5 E, Table III).

The peptidoglycan, which is a ligand for certain LysM domain proteins (WILLMANN et al, Proc Natl Acad Sci USA, 108, 19824-9) has also been tested in experiments of equilibrium binding, no competition with the radioactive LCO for binding to LYR3 was observed (results not shown).

### EXAMPLE 4

### Characterization of Lcos Binding Capacity of Protein Homologs of MtLYR3 in Legumes and Non Legumes

Closest counterparts of MtLYR3 were searched on the basis of sequence homology with the extracellular domain in legumes of agronomic interest (Pisum sativum, pea, Glycine max, soybean, Phaseolus vulgaris, French bean), and in the Lotus japonicus legume model. This research was extended to non-legumes whose genome is sequenced: Prunus persica (peach), Solanum lycopersicum (tomato) and Arabidopsis thaliana.

In legumes, MtLYR3 orthologs have been identified in P. sativum, P. vulgaris and L. japonicus. In G. max orthologs exist in duplicate due to a duplication of the genome. The corresponding genes have been cloned and sequenced. A few differences in sequence compared with sequences in the databases were found which could be explained by the use of different varieties of plants or by sequencing errors in databases. The cloned genes were introduced into binary vectors, in C-terminal fusion with the fluorescent protein YFP, and introduced into Nicotiana benthamiana leaves using agroinfiltration by Agrobacterium tumefaciens, as described above in Example 3. The membrane fractions were isolated, their protein content was analyzed by electrophoresis under denaturing conditions, followed by immunodetection using anti-GFP antibody as described in Example 3, and their ability to interact with LCOs was determined by equilibrium binding, as described in Example 1, in the presence of 1 nM LCO-IV (S³⁵, C16:2Δ2, 9). The value of specific binding is determined by the value of difference between the total binding and the nonspecific binding obtained for incubation in the presence of an excess (2 μM) of unlabeled ligand. For comparison, the ability to interact of LjNFR5 and LjNFR1 receptors, identified in L. japonicus to play a role in nodulation by a genetic approach (MADSEN et al, Nature, 425, 637-40, 2003. RADUTOIU et al., Nature, 425, 585-92, 2003a) and respective orthologs of LYK3 and NFP in M. truncatula was determined under the same conditions.

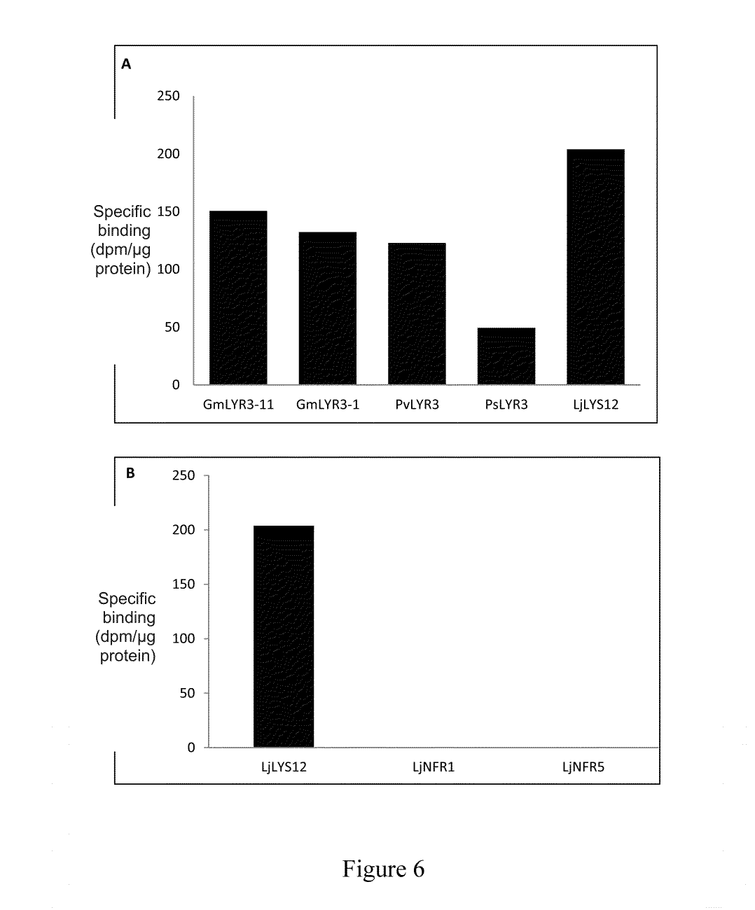

The results obtained are illustrated in FIG. 6, which shows the binding activity of membrane fractions from leaves of Nicotiana benthamiana expressing A) orthologs of LYR3 G. max (GmLYR3-1-11 and GmLYR3), P. vulgaris (PvLYR3, P. sativum (PsLYR3) and L japonicus (LjLYS 12) B) LjLYS12, LjNFR5 and LjNFR1.

These results show that all of the orthologs of MtLYR3 interact with LCO-IV (³⁵S, C16:2Δ2, 9). In contrast, NFR1 and NFR5 receptors of L. japonicus show no interaction with this LCO.

To further characterize the binding properties of MtLYR3 orthologs to LCOs, saturation and competitive binding experiments in the presence of different COs or LCOs were performed. The results are shown in Table IV below.

TABLE IV

| | Nod factors | | Myc-LCOs | | | |
|---|---|---|---|---|---|---|
| | LCO-IV | LCO-V | LCO-IV | LCO-IV | LCO-IV | COs |
| | (S,C16:2Δ2,9) | (S,C18:1Δ11) | (S,C16:0) | (C16:0) | (S,C18:1Δ9) | CO-V |
| | Affinity (K_{d}) | Affinity (Kᵢ) | | | | |
| PsLYR3 | 31 nM | 11 nM | 24 nM | 50 nM | 16 nM | >10 μM |
| PvLYR3 | 40 nM | 9 nM | 17 nM | 23 nM | 13 nM | >10 μM |
| GmLYR3-11 | 64 nM | 18 nM | 32 nM | 140 nM | 19 nM | >10 μM |
| GmLYR3-1 | 32 nM | 5 nM | 18 nM | 70 nM | 12 nM | >10 μM |
| LjLYS12 | 33 nM | 8 nM | 11 nM | 42 nM | 7 nM | >10 μM |

These results show that all orthologs have a high affinity, very close to that of MtLYR3, and comparable to the Myc-LCOs and the Nod factor LCO-IV (S, C16:2Δ2, 9). They confirm that LYR3 proteins recognize specifically the lipo-chitooligosaccharidic structure since the COs have a low affinity.

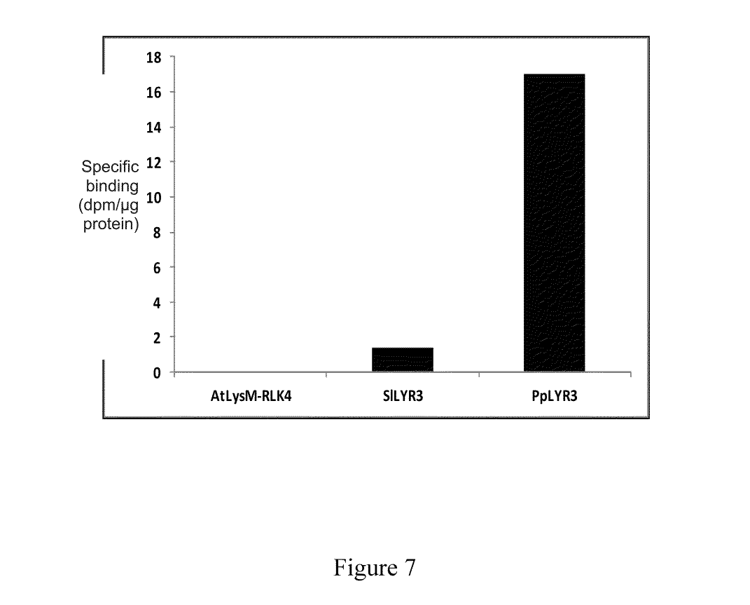

In non-legumes, PpLYR3, SILYR3 and AtLysM-RLK4 genes have been cloned, the corresponding proteins were expressed in N. benthamiana leaves, and their ability to bind to LCOs was determined by binding experiments at equilibrium, as described above for legumes LYR3 proteins. The results are illustrated in FIG. 7, which shows the binding activity of membrane fractions from leaves of Nicotiana benthamiana expressing PpLYR3, SILYR3 or AtLysM-RLK4 with Nod-factor LCO-IV (³⁵S, C16:2Δ2, 9). These results show that the protein of P. persica (PpLYR3) interacts with the tested LCO. In contrast, the interaction of the LCO with SILYR3 appears very low, and no interaction with the membrane fractions prepared from leaves expressing AtLysM-RLK4 could not be detected.

Since this apparent absence of binding may arise from the rapid dissociation of the interaction, due to a low affinity of these proteins for Nod factor, additional experiments using photoaffinity labeling were performed. The proteins, previously immuno-purified from extracts of N. benthamiana using an anti-GFP conjugated with magnetic beads (ChromoTek, Germany) antibodies were incubated in the presence of an analog containing a photoactivatable azido group, using the protocol described in Example 2 above.

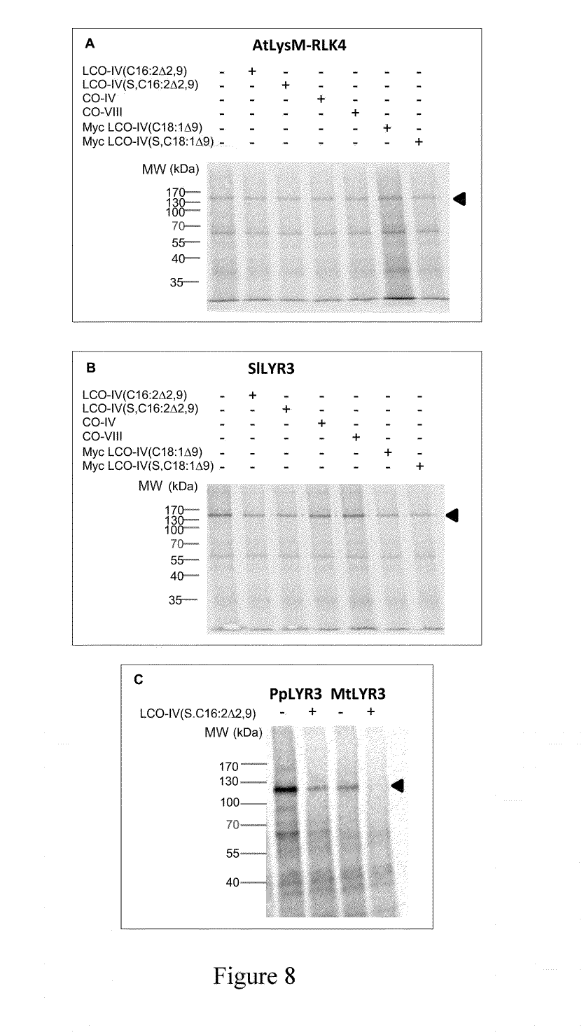

The results of this photoaffinity labeling are illustrated in FIG. 8. A: AtLysM-RLK4; B: SILYR3; C: PpLYR3 and MtLYR3. The competitor used is indicated above the corresponding track by the presence of a + sign.

For AtLysM-RLK4 (FIG. 8 A), the photoaffinity labeling does not appear specific since it is also observed in the presence of excess of competitor whether LCOs (Myc and Nod-LCOs factors) or COs (short or long chains). AtLysM-RLK4 seems devoid of ability to interact with the LCOs. For SILYR3 (FIG. 8B), the interaction is partially inhibited by an excess of LCOs but not by an excess of COs, suggesting that this protein recognizes lipochitooligosaccharides but with a low affinity and a selectivity different from MtLYR3 and its orthologs.

For PpLYR3 and MtLYR3 (FIG. 8C), the interaction with the photoactivatable derivative appears specific, because it is inhibited when the incubation is performed in the presence of an excess of LCO competitor.

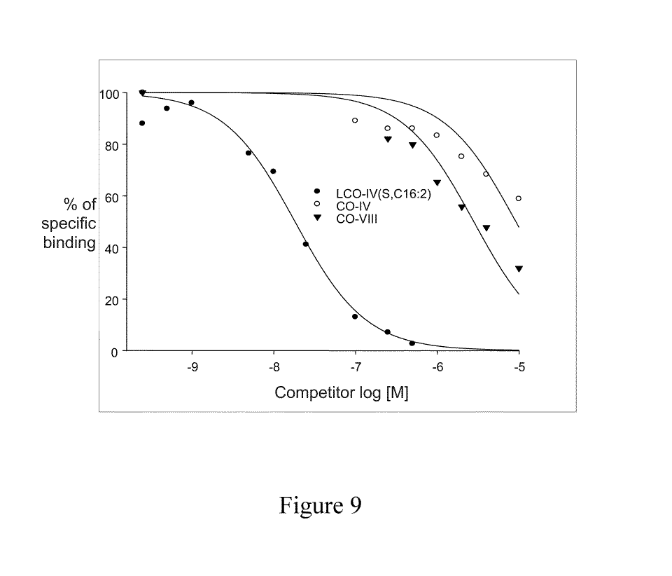

The interaction of PpLYR3 with the Nod LCO-IV (³⁵S, C16:2Δ2, 9) factor has been characterized in more detail by means of saturation and competition experiments. The results obtained are illustrated in FIG. 9. These results show that PpLYR3 exhibits high affinity for the Nod factor (Kd=15 nM) comparable to the affinity of MtLYR3. Similarly, PpLYR3 specifically recognizes the LCOs as interaction with COs (CO-IV and CO-VIII) is of low affinity (Ki>5 μM).

### EXAMPLE 5

### Creating Chimeric Proteins Containing the Extracellular Domain of LYR3 and Application for the Establishment of Plants with Defense Reactions Inducible by the LCOs

LysM-RLK1 or Chitin elicitor Receptor Kinase 1 (AtCERK1) is a major player in the perception of chitin in A. thaliana (MIYA et al, Proc Natl Acad Sci USA 104: 19613-19618; WAN et al, Plant Cell. 20:471-481; IIZASA et al, J Biol Chem 285:2996-3004; PETUTSCHNIG et al, J Biol Chem 285 (37):28902-28911). The perception of the chitin by the AtCERK1 extracellular domain and its transduction via the kinase and transmembrane domains, result in rapid cellular responses such as the production of reactive oxygen species, variations in the content of calcium or cytosolic pH intracellular, leading to the activation of defense genes. In order to create plants with defensive reactions that could be induced via the interaction of LCOs with the extracellular domain of LYR3, constructs allowing the production of chimeric proteins, consisting in the extracellular domain of LYR3 (LYR3-ED) fused to a transmembrane domain (TM) and to the intracellular kinase domain (Kin) of AtCERK1 were generated. For these constructs, the cloning method “Golden Gate” was used (ENGLER et al, PLOS One, 3 (11). E3647, 2008) Chimeric genes LYR3-ED/AtCERK1-TM-Kin and LYR3-ED-TM/AtCERK1-Kin, having a double HA tag and Strep-tagll were inserted into a binary vector pCAMBIA2200 (http://www.cambia.org; GenBank: AF234313.1), as amended, under the control of the 35S promoter. A construct allowing the reconstruction of CERK1 gene from nucleotide sequences encoding its extracellular domain and its transmembrane domain fused to its intracellular kinase domain (TM-AtCERK1-ED/AtCERK1 Kin) was conducted using the same approach. These different constructs were introduced in Agrobacterium tumefaciens GV3101 and in Agrobacterium rhizogenes MSV440 to transform stably or transiently a cerkl mutant of A. thaliana expressing the aequorin calcium probe described by WAN et al. (Plant Physiology 160:396-406, 2012). Transformation protocols established by CLOUGH and BENT (The Plant Journal 16: 735-743, 1998) and MARION et al. (The Plant Journal 56: 169-179, 2008) were applied respectively. Transgenic plants obtained are used to monitor calcium changes resulting from the perception of LCOs by the chimeric protein and the implementation of defense reactions protein.

The EFR receptor is responsible for the perception of the EF-Tu (elongation factor thermo unstable) bacterial elicitor resulting, at the cellular level, for example, in the producing of ethylene or reactive oxygen species (ZIPFEL et al. Cell 125, 749-760, 2006). According to the same principle, constructs allowing the production of chimeric proteins consisting of the extracellular domain of LYR3 (LYR3-ED) fused to a transmembrane domain (TM) and to the intracellular kinase domain (Kin) of the EFR receptor were generated. Transgenic plants obtained can track, following the application of LCOs, the defense responses characteristics of the EFR receptor.

1) Use of a polynucleotide selected from:
a polynucleotide encoding a receptor kinase whose extracellular LysM motifs domain flanked by the signal peptide, has at least 45%, and by order of increasing preference, at least 50, 55, 60, 65, 70, 75, 80, 85, 90, 95, or 98% sequence identity, and at least 65%, and by order of increasing preference, at least 70, 75, 80, 85, 90, 95 or 98% sequence similarity with the region 1-274 of Medicago truncatula LYR3 protein sequence SEQ ID NO: 2;
a polynucleotide encoding a polypeptide comprising the extracellular domain of said LysM motifs receptor kinase;
to improve the response of a plant to at least one LCO selected from the Nod factors and the Myc-LCO factors.

2) A method for improving the response of a plant to at least one LCO selected from the Nod factors and the Myc-LCO factors, characterized in that it comprises transforming the said plant with a polynucleotide encoding a LysM motifs receptor kinase, or a polypeptide comprising the extracellular domain of said receptor, as defined in claim 1, and expressing of said receptor or said polypeptide in said plant.

3) An expression cassette comprising a polynucleotide encoding a receptor kinase with LysM motifs, or a polypeptide comprising the extracellular domain of said receptor, as defined in claim 1, placed under the transcriptional control of a suitable promoter.

4) A recombinant vector containing an expression cassette according to claim 3.

5) A host cell transformed with a polynucleotide encoding a LysM motifs receptor kinase, or a polypeptide comprising the extracellular domain of said receptor, as defined in claim 1.

6) A transgenic plant comprising in its genome at least one copy of a transgene comprising a polynucleotide encoding a LysM motifs receptor kinase, or a polypeptide comprising the extracellular domain of said receptor, as defined in claim 1.


SEQUENCE LISTING (12 sequences)

SEQ ID NO: 1 - DNA, 1914 bp, Medicago truncatula
  CDS (1)..(1914)
atgcatctctttcccttcatcccttttttcattttcaatctcttcttcttcttcttcgtcagcaacatctccatcatcctaggacaacaaccttatattggtttagcaacaaatgcatgtccacgaaaaggcgattcaaaatcaatccgaggttacacatgtaacggcaaaactcaaacatgccaagcatacctcaccttcagaactcaaccaatttactcctcagtttcaacaatatcatcattactaggctcaaatccatctcaactcgccgaaataaactccgtttctttaaacgaaacattcgaaacaaacaaaatggtaattgttcctgtcaattgttcttgttctggtaactattatcaagcaaatacatcctatgtttttcaaaacacagacacttattttatagttgctaacaacacttttgaaggactttcaacatgtcaagctttgatgcatgaaaatcataatcctggtgatgtatatcctggtagaaaactacttgttcctcttagatgtgcttgtcctacaaagaatcaaactcagaaaaacataaagtatcttttgagttatttggttgattggggtgattctgtttcattcattagtgataaatttggtgttaactttagaaccactcttgaagctaatacactttctttaacacaatctacaatttatccctttacaacacttcttgttcctctttttgataagccctcaagttctcaaattcaaacacatcattctccttcatcatccccacctttatcttcttcttcttcgaccgacaaaaaatcgaaaaaaacttgggtttatgttgttgttggagtacttggaggagttgtaattgtagctttaatattatttttatatgctttcatttccttcaaaaagggtaaaaagaaaaatgattttttggtgagtgtttctgaaagtactatttttgaggaaaaagagaaaccaatgaagaaagaagatgaaaaattgtctgagattatatgtggtatagctcaatctttcaaagtgtatgattttgaggaaattaaggttgcaactgataattttagtccaagttgtcgggttaaaggaactgtttatcgcggccttattaaaggcgatttggctgcgattaagaagacagaaggagatgtttcaaaagagatacaaattctaaacaaagttaaccattccaatgttattcgtctttccggtgttagcttcaatcaaggacattggtaccttgtttatgagtatgctgctaatggagcattaagtgattggttattttctaacaaaaaaatggatgatggaaatattctgagttggattcggagaataaagattgcattagatgtggcaataggagttgaatatcttcatagtttcacttctcctccacatattcataaggatctaaagtgtagtaacatacttcttgatagtgattttaaagcaaaggttgcaaatttaaggcatgtaaggtgtgtggaagaagttgaaaatgatgaagaatttgttgctacaagacatattgttgggacaagaggttacatggctcctgagtatttggaaaatggtcttgtttctacaaagcttgatgtgtatgcatttggtattttgatgttggaaattattacaggaaaagaggttggttttatgatatcaaaagataatgagaatttgttggatgttttgagtggaatacttggtgagaaaagtggtgatgagaagttgaaggagtttatggatccttcattgcaaggaaattatccatttgaacttgctatgtttgtgattgaaattattcagaattgtttaaacaaggatccaggaaatagacctgctatggatgagattgtaccagttttgtcaagaacattgaattcttcattgagttgggaaatgtaa

SEQ ID NO: 2 - protein, 637 aa, Medicago truncatula
MHLFPFIPFFIFNLFFFFFVSNISIILGQQPYIGLATNACPRKGDSKSIRGYTCNGKTQTCQAYLTFRTQPIYSSVSTISSLLGSNPSQLAEINSVSLNETFETNKMVIVPVNCSCSGNYYQANTSYVFQNTDTYFIVANNTFEGLSTCQALMHENHNPGDVYPGRKLLVPLRCACPTKNQTQKNIKYLLSYLVDWGDSVSFISDKFGVNFRTTLEANTLSLTQSTIYPFTTLLVPLFDKPSSSQIQTHHSPSSSPPLSSSSSTDKKSKKTWVYVVVGVLGGVVIVALILFLYAFISFKKGKKKNDFLVSVSESTIFEEKEKPMKKEDEKLSEIICGIAQSFKVYDFEEIKVATDNFSPSCRVKGTVYRGLIKGDLAAIKKTEGDVSKEIQILNKVNHSNVIRLSGVSFNQGHWYLVYEYAANGALSDWLFSNKKMDDGNILSWIRRIKIALDVAIGVEYLHSFTSPPHIHKDLKCSNILLDSDFKAKVANLRHVRCVEEVENDEEFVATRHIVGTRGYMAPEYLENGLVSTKLDVYAFGILMLEIITGKEVGFMISKDNENLLDVLSGILGEKSGDEKLKEFMDPSLQGNYPFELAMFVIEIIQNCLNKDPGNRPAMDEIVPVLSRTLNSSLSWEM

SEQ ID NO: 3 - DNA, 1902 bp, Glycine max
  CDS (1)..(1902)
atggatctctttcccttcatccccatcatcattttcacactattgatccacaacttctctctgattctggggcagcaaccttacattggtttaggcacagtagcgtgcccaagaaggggtaacaaaaattctatccgtggttacacttgcaatggtgcaaaccatagctgccaaagttacctcaccttcagatctcaacccatctacaactctgtcaagacaatatcaactttgttgggttctgacccatcccagcttgctaaaataaactcagtttccatgaatgacacctttgaaacaaacaagttggttattgttccggtcaactgttcctgtgcaggtgagtattatcaaacaaacacatcctatgagttccataattcagaaacttacttcttgattgccaacaatacttttgagggcctcacaacatgccaagctttggagaaccaaaaccacaaccctgcaaacatataccctggtagaaggcttttagtgcctcttagatgtgcttgtcccacaaagaatcaaactgagaaaggcatcaggtacctcctaagttacttggtaaactggggtgattctgtttcattcattagtgagaaatttggtgtcaactttatgaccactcttgaagctaatacacttactctcacccaagccacgatctatccctttaccacaattctagttccccttcatgacaagccctcaagttctcaaactgtttcgccaactcggcgcactccaccaccctctcctccctcttctgatcatagctcaaacaaaacatgggtgtatgtagttgttggggttgttgtgggagctattgccttaatatcggttctctgtgctgtcattttcttcacacgctatcgcaaaaatagaaagaaagatgactcagtggtagtagggtccaagagttttgaggcaattgaggaaaaaccagaagtgaaagtgaatgaaaaattgtcagagatcatatctggcatagctcagtctttcaaagtgtataattttgaggaactacagcgtgcaacagataactttagtcctagcagctggatcaaagggtctgtttatcgcggtgtgattaacggtgatttggctgcaattaaaaggatagaaggagatgtgtcaaaagagatagagatactgaacaaaatcaaccattccaatgttatacgcctttccggggttagcttccacgaggggggttggtaccttgtttatgagtatgccgctaatggggacttgagtgaatggatctacttccacaacgtgaatgggaaatttctgagttggacgcagagaatgcagattgcattggatgtggccacaggacttgactatcttcacagtttcacttctcctcctcatatccacaaggatataaacagcagtaacattcttctggatggtgatttcaggggaaaggtcacgaatttaagccttgctaggtgtttggaaggaggggacgatcaacttcccgcgacgaggcacattgttgggacaagaggctacatggctccagagtatttggaaaatggtcttgtgtctacaaagcttgatgtgtatgcatttggggtacttatgctggaaatggtcactggaaaagaggttgctgctattttaactgaagatgagacaaaattgtcacatgttttaagtggcatacttggtgaggaaagtggcaaggagatgttgaaggagtttgtggatccctctttgggagaaaattgtccattggaacttgctatgtttgtgattgaaatgattgataattgcataaagacagatccagcaagtcgccctagtgtgcatgagattgtgcaatctctttcaagaacagtgaactcttcactgagttgggaaaggtcaatgaatgtcccacgaaattaa

SEQ ID NO: 4 - protein, 633 aa, Glycine max
MDLFPFIPIIIFTLLIHNFSLILGQQPYIGLGTVACPRRGNKNSIRGYTCNGANHSCQSYLTFRSQPIYNSVKTISTLLGSDPSQLAKINSVSMNDTFETNKLVIVPVNCSCAGEYYQTNTSYEFHNSETYFLIANNTFEGLTTCQALENQNHNPANIYPGRRLLVPLRCACPTKNQTEKGIRYLLSYLVNWGDSVSFISEKFGVNFMTTLEANTLTLTQATIYPFTTILVPLHDKPSSSQTVSPTRRTPPPSPPSSDHSSNKTWVYVVVGVVVGAIALISVLCAVIFFTRYRKNRKKDDSVVVGSKSFEAIEEKPEVKVNEKLSEIISGIAQSFKVYNFEELQRATDNFSPSSWIKGSVYRGVINGDLAAIKRIEGDVSKEIEILNKINHSNVIRLSGVSFHEGGWYLVYEYAANGDLSEWIYFHNVNGKFLSWTQRMQIALDVATGLDYLHSFTSPPHIHKDINSSNILLDGDFRGKVTNLSLARCLEGGDDQLPATRHIVGTRGYMAPEYLENGLVSTKLDVYAFGVLMLEMVTGKEVAAILTEDETKLSHVLSGILGEESGKEMLKEFVDPSLGENCPLELAMFVIEMIDNCIKTDPASRPSVHEIVQSLSRTVNSSLSWERSMNVPRN

SEQ ID NO: 5 - DNA, 1905 bp, Glycine max
  CDS (1)..(1905)
atggatctctttcccttcatccccataatcattttcacactattgatccacaacttctctctgattctggggcagcaaccttatattggtttaggcacagtagcgtgtccaagaaggggtaacaaaaattctatccgaggttacacttgcaatggtgcaaaccatagctgccaaagttacctcaccttcagatctcaacccatttacaactctgtcaagacaatatcaactttgttgggttctgacccatcccagcttgctaaaataaactcagtttccatgaatgacacctttgagacaaacaagttggtgattgttccggtcaactgttcctgttcaggtgagtattatcaaacaaacacatcctatgtgttccagaattcagaaacttacttgttgattgctaacaacacttttgagggcctcacaacatgtcaagctttggagaaccaaaaccacaaccctgcaaacatataccccggtagaagacttttagtgcctctcagatgtgcttgtcccacaaagaaccaaaccaagaaaggcatcaggtacctcttgagttacttggtgaactggggtgattctgtttcattcattagtgagaaatttggtgtcaactttatgtctactcttgaagctaatacccttactctcacacaagccatgatctatccctttacgacaattttagttccccttcatgacaagccctcaagttctcaaaccgtttcgccaactcaacgcattagtccaccaccctcacctccctcttctgatcatagctcaaacaaaacatgggtgtatgtagttgttggggttgttgtgggagctattgccttaacatcggttctctgtgctgtcattttcttcaaacgctatcgcaaaaatagaaacaaagatgactcattggtggcagtgcctaagagttttgaggcaattgaggaaaaacctcaagtgaaagtgaatgaaaaattgtcagagaacatatctggcatagctcagtctttcaaagtgtataactttgaggaactacagcgtgcaacagataactttagtcctagcagctggatcaaagggtctgtttatcgcggtgtgattaatggtgatttggctgcaattaaaaagatagaaggagatgtgtcaaaagagatagagatactgaacaaaatcaaccataccaacgttatacgcctttctggagttagcttccatgagggtcgttggtaccttgtttatgtgtatgctactaatggggacttgagtgaatggatctacttcaacaacgtggacgggaagtttttgagttggactcaaagaatgcaaattgcattggatgtggcaacaggacttgactatcttcacagtttcacttctcctcctcacatccacaaggatattaacagcagtaacattcttctggatggtgatttcaggggaaaggtcgcgaatttaagccttgctaggtgtttggaaggaggggatgatcaatttcccacgacgaggcacattgttgggacaagaggctacatggctccagagtatttggaaaatggtcttgtgtccacaaagcttgacgtatatgcatttggggtactgatgctggaaatggtcactggaaaagaggtcgctgctattttaactgaagatgagacaaaattgtcacatgttttaagtggcatacctggtgagagaagtggcaaggagtggttgaaggagtttgtggatccctctttgggagagaattgtccattggaacttgctatgtttgtgattgaaatgattgatgattgcataaagacagatccagcaagtcgccctagtgtgcatgagattgtgcaatctctttcaagaacagtgaactcttcactgagttgggaaaggtcaatgaatgtcccacgaaattaa

SEQ ID NO: 6 - protein, 634 aa, Glycine max
MDLFPFIPIIIFTLLIHNFSLILGQQPYIGLGTVACPRRGNKNSIRGYTCNGANHSCQSYLTFRSQPIYNSVKTISTLLGSDPSQLAKINSVSMNDTFETNKLVIVPVNCSCSGEYYQTNTSYVFQNSETYLLIANNTFEGLTTCQALENQNHNPANIYPGRRLLVPLRCACPTKNQTKKGIRYLLSYLVNWGDSVSFISEKFGVNFMSTLEANTLTLTQAMIYPFTTILVPLHDKPSSSQTVSPTQRISPPPSPPSSDHSSNKTWVYVVVGVVVGAIALTSVLCAVIFFKRYRKNRNKDDSLVAVPKSFEAIEEKPQVKVNEKLSENISGIAQSFKVYNFEELQRATDNFSPSSWIKGSVYRGVINGDLAAIKKIEGDVSKEIEILNKINHTNVIRLSGVSFHEGRWYLVYVYATNGDLSEWIYFNNVDGKFLSWTQRMQIALDVATGLDYLHSFTSPPHIHKDINSSNILLDGDFRGKVANLSLARCLEGGDDQFPTTRHIVGTRGYMAPEYLENGLVSTKLDVYAFGVLMLEMVTGKEVAAILTEDETKLSHVLSGIPGERSGKEWLKEFVDPSLGENCPLELAMFVIEMIDDCIKTDPASRPSVHEIVQSLSRTVNSSLSWERSMNVPRN

SEQ ID NO: 7 - DNA, 1851 bp, Phaseolus vulgaris
  CDS (1)..(1851)
atgcatctctttcccttcactctcataatctttttcaccgttttgatccacaacttctctctgattctggggcagcaaccttatattggtttaggcacagtagcatgcccttcaagtggtaacaaaaactctattcgtggttacacttgtaatggtgcaaacagctgccaagcattccttactttcagatctcaacccatttacaactctgtctccacaatatctactttattaggttctgatccgtcccaacttgctcatataaattccgtttccctgaatgacaccttccaaacaaacaagttggtgcttgttccggtcaactgttcctgtgcaggtgagtattatcaaacaaacacatcctatgtattccagaattcagaaacttacttcttgattgctaataacactttcgagggcctcacaacatgtcaagctttgcagagtcaaaaccacaacccagcaaacatataccccggtagaaaacttctagtgcctctcagatgtgcttgtcccaccaaaaaccaaaccgagatagccattaggtacctcttaagctacttggtgaagccggatgattttgtttcagtcatcgctgagaaatttggtgttggcactatgaccactcttgaagctaatagcctcactttgtcacaatccactatctatccctttactacacttctaattccccttcacgataagccctcaagttctcaaaccgtttctccagctcaacttacgccacccccctcatcacccccctcttctcatcacaagaaaacatgggtgtatgctgttgttggagctgttgccttgacatcagctctttgtgctgtcattttcttcacacgctatcgcaaaaatagaaacaaagacgagtcagcggtagtggccaagaattttgaggcaactaagaaaaaaccacaaaggagggtaaatgaaaaattttcagagatcatatctggcataggtcaatcgttcaaggtgtatgattttgaggaactaaagcgagcaacagataactttagtcctagctactggatcaaagggtctgtttatcgcggtgtgattaatggtgatttggcagcaattaaaaagatcgaaggtgatgtgacaaaggagatggagatactgaacaaaatcaaccattccaatgttatacgcctttccggggttagcttccacgaggggcgttggtaccttgtttttgagtatgctgctaatggagctttgagtgaatggatcttcttcaacaacgtggatgggaagtttctgagttggacgcagagaatacagattgcattggatgtggccacaggactagattatcttcacagtttcacttctcctccttatatccacaaggatatcaagagttgtaacattcttctggatggtgacttcaggggaaaggttgcaaatttaagccttgttaggcgtgtggaaggaggggaagatcaatttcctgccactaggcacattgttgggacaagaggctacatggctcccgagtatttggaacatggtcttgtatctacgaagcttgatgtgtatgcttttggggtagtgatgctggaaatgatcacaggaaaagaggttgctgctattctaacagaagacgagacaaacttgtcacatgttttacgtgtcatgcttggtgaagaaagtggccaggagaggttgaaggcgtttgtggatccctctttgcaagaaaattgtccatttgaacttgctatgtttgtgattgaaatgattgataactgcatatcgagtgatccagcaagtcgtcctagtgtgcatgagattgtgcaatctctgtcaagaaccctgaattcttcactgagttag

SEQ ID NO: 8 - protein, 616 aa, Phaseolus vulgaris
MHLFPFTLIIFFTVLIHNFSLILGQQPYIGLGTVACPSSGNKNSIRGYTCNGANSCQAFLTFRSQPIYNSVSTISTLLGSDPSQLAHINSVSLNDTFQTNKLVLVPVNCSCAGEYYQTNTSYVFQNSETYFLIANNTFEGLTTCQALQSQNHNPANIYPGRKLLVPLRCACPTKNQTEIAIRYLLSYLVKPDDFVSVIAEKFGVGTMTTLEANSLTLSQSTIYPFTTLLIPLHDKPSSSQTVSPAQLTPPPSSPPSSHHKKTWVYAVVGAVALTSALCAVIFFTRYRKNRNKDESAVVAKNFEATKKKPQRRVNEKFSEIISGIGQSFKVYDFEELKRATDNFSPSYWIKGSVYRGVINGDLAAIKKIEGDVTKEMEILNKINHSNVIRLSGVSFHEGRWYLVFEYAANGALSEWIFFNNVDGKFLSWTQRIQIALDVATGLDYLHSFTSPPYIHKDIKSCNILLDGDFRGKVANLSLVRRVEGGEDQFPATRHIVGTRGYMAPEYLEHGLVSTKLDVYAFGVVMLEMITGKEVAAILTEDETNLSHVLRVMLGEESGQERLKAFVDPSLQENCPFELAMFVIEMIDNCISSDPASRPSVHEIVQSLSRTLNSSLS

SEQ ID NO: 9 - DNA, 1938 bp, Pisum sativum
  CDS (1)..(1938)
atgcatctctttcctttcattttcactctcttcttcttcttcttcttcatcaccaacatctccatcatcctaggacaacaaccttacattggcctatccacaacagcctgtcctcgaaaaggcgactcgaaatcaatccgtggctacacatgtaacggccaaactcacacatgccaagcctacctcaccttcagatctcaaccaatttacagttcagtttcaacaatctcatctttactaggctcaaacccttctcaactcgcagaaattaactcggtttctgtaaacgaaaccttcgaagtaaacaaaatggtaattgttcctgtcaactgttcttgttctggtagctattaccaagcaaacacatcctatgtttttcaaaacacagacacttatttcttagttgctaacaacactttcgaaggcctttccacttgtcaagctttgatgcatgaaaatcataaccctgctgatatatatcctggtagagaattacttgttcctcttagatgtgcttgtcctacaaagaatcaaactgaaaacaacattaagtatctcttaagctttttggttaattggggtgattcagtttcactcattagtgaaaaatttggtgttaacttcaaaaccactcttgtagctaatactcttactttaacacaagctactatttatccttttacaacacttcttgttcctctttttgataagcctaaaagttctcaaattcaaacagtttcggtttcgccacatcaacaaccttctccttcatcaacctcgccttcatcttcttcttcttcgaccgatagaaaatcgggaaaaactttggtttatgttgtggttggagtacttggaggacttgtagttattatagctttaatgttaattctatttgctttaattttctttaaaaagggtaaaaagaaagatgatttttcggcgaatgtttcggagagtattatttattcggcgaaagagaaacctgtgaagaaagaagatgaaaagttattagagattatatctggtataggccaatctttcaaagtgtatgattttgaagaaattaaggttgcaacagatgattttagtccatgttttttgatcaaaggttgtgtttatcgcggtgttattaacagcgatttggctgcaattatgaaaacagaaggagatgtttcacaagagatacagattctcaacaaagttaaccattctaatgttattcgtctttcgggtgttagcttcaacgaaggacattggtatcttgtttatgagtatgctgcaaatggatcgttaagcgattggatattttctaaccataaaatgagtgatgaaaagtttctgagttggattcagagaatgaagattgcattggatgttgcaacaggagttgaatatcttcatagtttcacttcacctccacatgttcataaggatcttaaatgtagtaatgttcttctcgacaacgaattcaaggcgaaggttgcgagtttaaggcatgcgaggtgtgtggaaggatttgaagatgatgatgatgaagaatttgttgctacaagacatattgttgggacaagaggttacatggcaccggagtatttggaaaatggaatcgtttctacgaagctcgacgtttatgcgttcggtgtactgatgttggaaatcatcacagggaaagaggttgctgctattttatcagaagataatgagaatctgatagacttcttaaagggtgtagttgatgaggagaatgataatgagaagttgaaggagttaatggattcttcgttgcaaggaaattatccatttgagcttgctatgtttgtgattgaaattattgagagttgtttaaagaaggatccgggaaatcgtcctgcgatggatgaggttgtatcggctttgtcaagaacattgaactcttcagtgaatgttggtagatactaa

SEQ ID NO: 10 - protein, 645 aa, Pisum sativum
MHLFPFIFTLFFFFFFITNISIILGQQPYIGLSTTACPRKGDSKSIRGYTCNGQTHTCQAYLTFRSQPIYSSVSTISSLLGSNPSQLAEINSVSVNETFEVNKMVIVPVNCSCSGSYYQANTSYVFQNTDTYFLVANNTFEGLSTCQALMHENHNPADIYPGRELLVPLRCACPTKNQTENNIKYLLSFLVNWGDSVSLISEKFGVNFKTTLVANTLTLTQATIYPFTTLLVPLFDKPKSSQIQTVSVSPHQQPSPSSTSPSSSSSSTDRKSGKTLVYVVVGVLGGLVVIIALMLILFALIFFKKGKKKDDFSANVSESIIYSAKEKPVKKEDEKLLEIISGIGQSFKVYDFEEIKVATDDFSPCFLIKGCVYRGVINSDLAAIMKTEGDVSQEIQILNKVNHSNVIRLSGVSFNEGHWYLVYEYAANGSLSDWIFSNHKMSDEKFLSWIQRMKIALDVATGVEYLHSFTSPPHVHKDLKCSNVLLDNEFKAKVASLRHARCVEGFEDDDDEEFVATRHIVGTRGYMAPEYLENGIVSTKLDVYAFGVLMLEIITGKEVAAILSEDNENLIDFLKGVVDEENDNEKLKELMDSSLQGNYPFELAMFVIEIIESCLKKDPGNRPAMDEVVSALSRTLNSSVNVGRY

SEQ ID NO: 11 - DNA, 1983 bp, Prunus persica
  CDS (1)..(1983)
atgagtttcctctctctcatcttatctgccatcattctcaccactttcagttgctgttctttgattgttgctcagcagccttatgagggcaaggcgtttacagattgttacaacacaaacacttactcaaaatctgttcttgggtacagctgcaatggagtgaacaaaagttgccaaacttacctcaccttcagatcccagcctccttacaacagtgtttctgcaatctctgctatgttggcttctgacccgtctcagattgcggagatgaattcggtttccgagactgcaacgtttgaaacgaacaagttggtgattgttccaatcacatgctcctgctcaggtgatttctatcagctaaacacatctcatgtagttgtgcatggtgatacatattttgtgattgcaaataacactttgcaaggcctctcaacatgtcaagctatgatgaaccagaatactaatctcactacaaaagagttgtattctggtactagacttagtgttcctcttagatgtgcttgtcccacaaagaaccaaactgatgtggggaccaagtatcttttgacttacataatcgctcaaggggattatgtttcaaagattagtgctacatttgattcggataccggaagaactcttgaagctaatgggctttctgagcaagcctctaccatttatcccttcaccacacttttagttcctttgcaaaaccctccatctagttctcagaccatagagccaccctcatcatcaccatcacctccggcgccaccttcaacttcctctaataactcaacgaaaaaatgggtttatgcctttgttggggctcttggaggaggtgcttttgtgttggtcataggcatcattatcttctgcacattctttcgcagaagcagaggtacgaagggtttcaattcaattattgtctcggagaggtttgaggcacttgagaaaccactagagaaaaaggaggaagaaggatctcaggacttcttagaggctctatctggcatagctcaatccctcaaagtttacaaatttgaggaactgcaacgcgcaacagatgactttagctccacttatttgattaaggggtctgtgtatcgtggaaacatcaatggggatttggcagccatcaagaaaatgaatggagatgtgtccacagagataaatttactaaacaaaaccagccattctaatctcatttgcctgtcaggagtttgctttcatgatggacattggtatcttatatatgagtatgctgtcaatggacccttgagtgattggatctattacagcaacaatgatgggaaatttctgaattggacacaaagaatacagattctgttggatgtggcctcagggcttaattatctccacagcttcaccactcctccccatgtccacaaggatataaagagcagtaacattcttcttgacagtgacttcaggggcaagattgcaaacctaggtctagcaaggtcaaccgaagcgccggaaggtgaggttcctttgacaaatcacattgttggaacaataggatacatggctcctgagtacttggagaatggtttgatctccacaaagcttgatgtctatgcatttggggctctcatgctagaaatactcactggaaaagaggttgctgtgctttatgaggaaaatagacaattgtctgatgtattaaactctgttctgaacgacgaagatggaccggagagtttgaggcacttcatcgatccttctatgcaagagagttatcctccggaacttgctctttttgtgatcagattggtccatagttgcttaaataaaaacccagctgctcgcccggcaatggaggagatggtacagtttctctcaagaactatgagcaattcactcagttgggaattgtcaagcaacatctcaggctaccaaggtacagagggatttcttgatggtagaggaactggcacagggtctcaatcaactacttag

SEQ ID NO: 12 - protein, 660 aa, Prunus persica
MSFLSLILSAIILTTFSCCSLIVAQQPYEGKAFTDCYNTNTYSKSVLGYSCNGVNKSCQTYLTFRSQPPYNSVSAISAMLASDPSQIAEMNSVSETATFETNKLVIVPITCSCSGDFYQLNTSHVVVHGDTYFVIANNTLQGLSTCQAMMNQNTNLTTKELYSGTRLSVPLRCACPTKNQTDVGTKYLLTYIIAQGDYVSKISATFDSDTGRTLEANGLSEQASTIYPFTTLLVPLQNPPSSSQTIEPPSSSPSPPAPPSTSSNNSTKKWVYAFVGALGGGAFVLVIGIIIFCTFFRRSRGTKGFNSIIVSERFEALEKPLEKKEEEGSQDFLEALSGIAQSLKVYKFEELQRATDDFSSTYLIKGSVYRGNINGDLAAIKKMNGDVSTEINLLNKTSHSNLICLSGVCFHDGHWYLIYEYAVNGPLSDWIYYSNNDGKFLNWTQRIQILLDVASGLNYLHSFTTPPHVHKDIKSSNILLDSDFRGKIANLGLARSTEAPEGEVPLTNHIVGTIGYMAPEYLENGLISTKLDVYAFGALMLEILTGKEVAVLYEENRQLSDVLNSVLNDEDGPESLRHFIDPSMQESYPPELALFVIRLVHSCLNKNPAARPAMEEMVQFLSRTMSNSLSWELSSNISGYQGTEGFLDGRGTGTGSQSTT